(19) United States
(12) Patent Application Publication
(10) Pub. No.: US 2026/0318827 A1
(43) Pub. Date: Oct. 15, 2026

(54) SHORTWAVE INFRARED (SWIR) CAMERA DETECTION OF FREE WATER IN LIQUID

(71) Applicant: Saudi Arabian Oil Company, Dhahran (SA)

(72) Inventors: Saidkhodzha Nematulloev, Thuwal (SA); Maha Nour, Thuwal (SA); Ali Al Shehri, Thuwal (SA); Hamad Alsaiari, Thuwal (SA); Diki Andrian, Ras Tanura (SA); Ali Saad Aldossary, Al Khobar (SA)

(21) Appl. No.: 19/178,253

(22) Filed: Apr. 14, 2025

(51) Int. Cl. G01N 21/3554; G01N 21/3577; G01N 21/85; G01N 21/88; G01N 33/28; G08B 21/20

(52) U.S. Cl. CPC G01N 21/3554; G01N 21/3577; G01N 21/85; G01N 21/8851; G01N 33/2847; G08B 21/20; G01N 2021/8557; G01N 2021/889; G01N 2021/8896

(57) ABSTRACT

Systems and methods for measuring water concentration in a liquid medium include a flow cell configured to fluidly couple to a pipe; a Shortwave Infrared (SWIR) camera coupled to a first side of the flow cell; a light source coupled to a second side of the flow cell opposite the first side; and one or more processor and a memory unit storing instructions that when executed by the one or more processors cause the one or more processors to perform operations. The operations include acquiring one or more SWIR images captured by the nanomaterial-based SWIR camera, applying a preprocessing operation to the one or more SWIR images; detecting water droplets in the one or more SWIR images using a trained machine learning model; determining a concentration of water in the liquid medium based on the detected water droplets; and in response, performing a mitigating action.

### TECHNICAL FIELD

This disclosure generally relates to a system and method for detecting and measuring free water concentration in a liquid medium.

### BACKGROUND

Oil and gas production involves extracting hydrocarbons from underground reservoirs, where they naturally coexist with water due to geological formation processes. During downstream processing of the extracted hydrocarbons, the water is separated from the hydrocarbons. As hydrocarbons move through extraction, transportation, and refining stages, separating water from the product becomes critical to prevent corrosion, equipment damage, and inefficiencies in downstream refining and petrochemical processes. Managing water content in oil and gas streams is important to ensuring operational reliability and product quality.

### SUMMARY

Free water in oil separates naturally due to gravity force in ambient conditions. However, measuring the free water (e.g., undissolved water) content in a liquid medium, such as hydrocarbon condensate or natural gas liquids (NGL), is challenging under inflow conditions due to the high flow rates, elevated pressure, high temperatures, and mixing values. As a result, free water droplets remain dispersed within the hydrocarbon condensate or NGL and are hard to monitor. Moreover, collecting samples for laboratory analysis is tricky and inconvenient due to water concentration changes that can occur in the collected samples due to evaporation at elevated temperature.

This disclosure describes systems and methods for detecting and measuring free water concentration in a liquid medium (e.g., hydrocarbon condensate, natural gas liquids). A SWIR camera is coupled to one side of the flow cell. A light source is coupled to a side of the flow cell opposite the SWIR camera. A data processing system (e.g., a computer or control system) acquires one or more images from the SWIR camera. The data processing system detects free water droplets in the SWIR images using a trained machine learning model. The data processing system determines a concentration of free water in the liquid medium based on the detected free water droplets.

Implementations of the systems and methods of this disclosure can provide various technical benefits. For example, the system enables the free water concentration to be determined in place without a need to take samples from a pipeline.

Additionally, the use of a SWIR camera leverages the distinct absorption features of water in the 1440-1930 nm range, which allows water to be clearly visible while other hydrocarbons remain transparent, enhancing the contrast between free water droplets and the liquid medium. Due to the clear contrast between a free water droplets generated in images between the free water droplets and the liquid medium, the trained machine learning model can detect the free water droplets with greater accuracy. The system enables high-precision detection of free water droplets in hydrocarbon condensate and NGL streams, even at low concentrations, for example, as low as 20 parts per million (ppm). The water concentration of the liquid medium can be monitored on a continuous basis by acquiring and processing time-series of images from the SWIR camera.

Unlike systems which solely use Charge-Coupled Device (CCD) sensors, SWIR sensors offer several advantages, including a more compact design, lower cost, higher resolution (e.g., up to 10 megapixels), a broader spectral range, improved photoelectric properties such as higher external quantum efficiency (EQE), and a reduced need for cooling.

Detection of water concentration above a threshold value can lead to early mitigation of environmental conditions that could lead to corrosion or damage of the pipeline or other equipment. In addition to measuring free water concentrations in hydrocarbon condensate, the systems and methods of this disclosure can be adapted for other applications, for example, water in oil, oil in water, or for monitoring concentrations of metals in a liquid medium.

The details of one or more implementations of these systems and methods are set forth in the accompanying drawings and the description below. Other features, objects, and advantages of these systems and methods will be apparent from the description and drawings, and from the claims.

### DESCRIPTION OF DRAWINGS

FIG. 1 is a schematic diagram of an example system for measuring free water concentration in a liquid medium.

FIG. 2 is a schematic diagram of another system for measuring free water concentration in a liquid medium.

FIG. 3 is a flowchart that represents an example free water detection workflow.

FIG. 4 is a flowchart for an example method for measuring free water concentration in a liquid medium using the systems of FIG. 1 or 2.

FIG. 5 is a block diagram illustrating an example computer system used to provide computational functionalities associated with described algorithms, methods, functions, processes, flows, and procedures according to some implementations of the present disclosure.

Like reference symbols in the various drawings indicate like elements.

### DETAILED DESCRIPTION

This specification describes systems and methods for detecting and measuring free water concentration in a liquid medium. A system includes a flow cell configured to be coupled to a pipe carrying the liquid medium (e.g., hydrocarbon condensate, natural gas liquids). A SWIR camera is coupled to one side of the flow cell. A light source is coupled to a side of the flow cell opposite the SWIR camera. A data processing system (e.g., a computer or control system) acquires one or more images from the SWIR camera. The data processing system detects free water droplets in the images using a trained machine learning model. The data processing system determines a concentration of free water in the liquid medium based on the detected free water droplets.

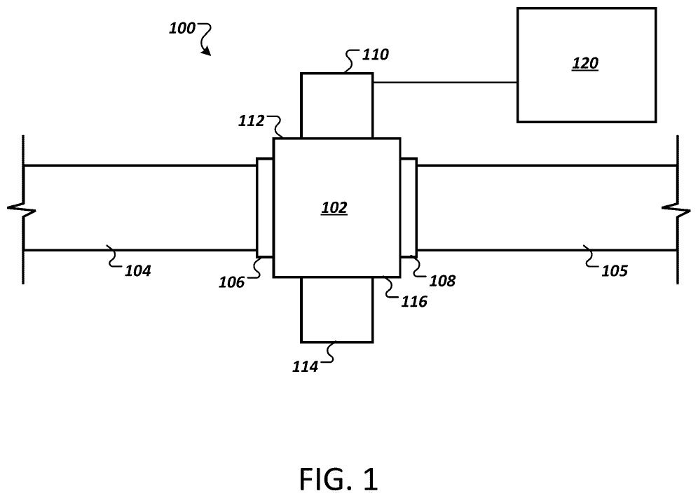

FIG. 1 is a schematic diagram of an example system 100 for measuring free water concentration in a liquid medium. System 100 includes a flow cell 102, which forms the measurement region for the system 100. The flow cell 102 serves as a conjunction between two pipes 104, 105, and is coupled in line with the two pipes 104, 105. The flow cell 102 is coupled to the pipe by flanges 106, 108.

A SWIR camera 110 is coupled to a first side 112 of the flow cell 102. The SWIR camera 110 includes a modular (e.g., rectangular) enclosure that houses a SWIR sensor, a passive cooling system, and filters and interchangeable lenses. In some embodiments, a nanomaterial-based SWIR sensor is used. For example, the nanomaterial-based SWIR sensor incorporate Indium Arsenide (InAs) or Lead Sulfide (PbS) quantum dots, which enable broad spectral sensitivity ranging from 800-2500 nm. The nanomaterial-based SWIR sensor exhibits high detectivity, enabling the detection of low concentrations of free water present in the liquid medium. It's low dark current minimizes noise in the captured images, particularly in low-light or high-flow conditions. The system 100 is capable of measuring low free water concentrations, for example, as low as 20 parts per million (ppm).

The passive cooling system surrounds the SWIR sensor, and dissipates heat generated during operation of the SWIR camera 110. The filters and interchangeable lenses are optimized for operation within the SWIR wavelength range (e.g., 900-2500 nm), and are positioned in alignment with the optical path of the sensor. The filters are designed to isolate specific wavelengths corresponding to water absorption features. The filters may include, but are not limited to, bandpass filters, long-pass filters, and short-pass filters. The interchangeable lenses are adapted for high transmission in the SWIR spectrum. The lenses may be interchanged based on the needs of a particular application. For example, when the SWIR camera 110 is equipped with a microscope objective lens, the system 100 can detect micro-scale free water droplets.

A light source 114 (e.g., a SWIR light source) is coupled to a second side 116 of the flow cell. The light source 114 emits wavelengths in the range of 700 to 2000 nm. [0001] The first side 112 and the second side 116 are opposite one another on the flow cell 102. The first side 112 and the second side 116 are transparent or include transparent portion to enable the SWIR camera 110 and the light source 114 to transmit and receive light through the flow cell 102. In some implementations, magnifying lenses are coupled to the transparent portions to magnify the flow of the liquid medium through the flow cell 102.

Liquid flowing through pipe 104 enters the flow cell 102 and exits into pipe 105. The liquid can be, for example, hydrocarbon condensates, C3+ NGL, or both. C3+ NGL includes natural gas liquids with molecules having three or more carbon atoms, e.g., propane, butane, pentane, hexane, heptane, and so forth. The carbon atoms can be single, double, or triple bonded or other arrangement (e.g., alkanes, alkenes, alkynes, cycloalkanes, alkadienes). The liquid medium flowing through the pipes 104, 105 can have elevated pressure (e.g., 400 psi or more) and temperature (e.g., 180 degrees Fahrenheit (°F) or more) relative to typical atmospheric conditions. The elevated temperature and pressures in the pipes 104, 105 make it impractical to take samples from either pipe to measure the free water concentration as the water can evaporate at the higher temperature at atmospheric pressure. The liquid can also have high flow rate (e.g., 60 million barrels per day (MBPD)) The high flow rate of the liquid can contribute to mixing of the liquid medium with the water and breakup of water droplets into smaller water droplets.

The system 100 also includes a data processing system 120 communicatively coupled to the SWIR camera 110. The data processing system 120 includes at least one processor and memory storing instructions for detecting and measuring the free water concentration in the liquid medium. The data processing system 120 can be for example a controller or computer. The data processing system is communicatively coupled to the SWIR camera or other components of the system 100 via wired (e.g., ethernet, serial communication link, USB) or wireless connections (e.g., Wi-Fi, Bluetooth, cellular). The data processing system can be located near to the measurement location or remote from the measurement location.

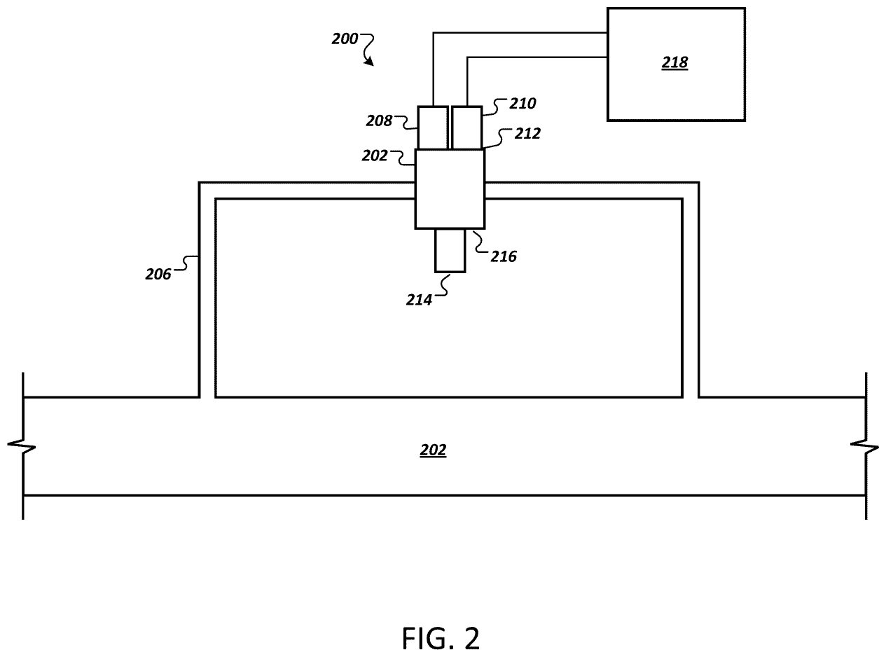

FIG. 2 is a schematic diagram of another example system 200 for measuring free water concentration in a liquid medium. System 200 includes a flow cell 202 configured to receive the liquid medium from pipe 204 via inflow loop 206. An inflow loop 206 can be used, for example, in systems having a pipe with a large diameter (e.g., 1 inch or more, 5 inches or more, 10 inches or more). The inflow loop 206 has a smaller diameter than the pipe 204. A SWIR camera 208 and a visible light camera 210 are coupled to a first side 212 of the flow cell 202. The visible light camera 210 includes a CCD sensor. A light source 214 is coupled to a second side 216 of the flow cell 202. The second side 216 is opposite the first side 212. The light source 214 is operable to emit light in the visible spectrum and in the SWIR spectrum.

The SWIR camera 208 and the visible light camera 210 are communicatively coupled to a data processing system 218. The data processing system 218 acquires images from both the nanomaterial-based SWIR camera 208 and the visible light camera 210, captured simultaneously with the same or overlapping fields of view, and processes them together to enhance free water detection accuracy.

In some implementations, a system for measuring free water concentration in a liquid medium can include features of system 100, system 200, or both. For example, a system coupled to a pipeline (as shown in FIG. 1) can include both a visible light camera and a SWIR camera. Likewise, a system coupled to an inflow loop (as shown in FIG. 2) can include a SWIR camera without including a visible light camera.

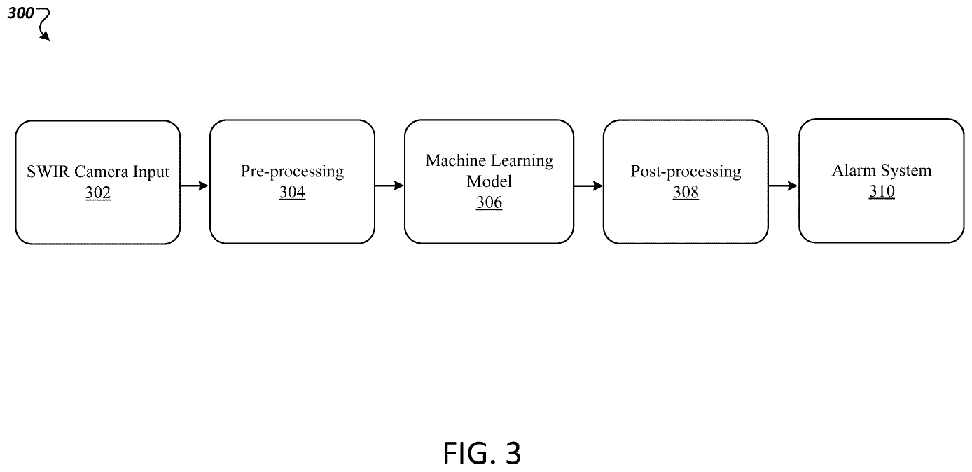

FIG. 3 is a flowchart that represents an example free water detection workflow 300. The free water detection workflow 300 can be implemented using a computing system, such as data processing system 120 or data processing system 218.

The data processing system obtains a sequence of SWIR camera input 302 captured by a SWIR camera. The SWIR camera input can include a sequence of SWIR images (e.g., time series of images) or SWIR videos depicting the liquid flowing through a pipe over a duration of time. The data processing system converts the SWIR video into a sequence of frames by extracting individual frames at specific intervals for further processing by the following steps.

The data processing system performs a pre-processing step 304 where the SWIR camera input is prepared for analysis by a machine learning model. The pre-processing step 304 includes removing unwanted artifacts from the SWIR images or frames, such as gas bubbles, solid particles, and noise. The data processing system may apply one or more pre-processing techniques, including but not limited to, contrast enhancement to improve image clarity, filtering techniques such as median and Gaussian filters to remove the noise, and morphological operations to refine the shape of detected water particles.

The data processing system provides the pre-processed SWIR camera input to the machine learning model 306 to calculate the free water concentration in the liquid medium. The machine learning model 306 processes the pre-processed SWIR images or frames to detect the free water droplets in the liquid medium and calculates the free water concentration based on the detected free water droplets. In some embodiments, the machine learning model 306 is implemented as a combination of a Convolutional Neural Network (CNN) and a Recurrent Neural Network (RNN). The CNN processes the SWIR images or frames to extract spatial features such as free water droplet shape and size, while the RNN processes the extracted spatial features as a sequence to identify temporal patterns in droplet movement or flow rate.

Specifically, the CNN processes a sequence of SWIR images or frames captured over a period of time, generating a plurality of spatial feature vectors, each spatial feature vector corresponding to a processed SWIR image or frame. The spatial feature vectors encode characteristics of detected free water droplets, such as shape and size.

The RNN receives the spatial feature vectors as a temporally ordered sequence to model temporal dependencies among them. The RNN may be implemented as a Long Short-Term Memory (LSTM) network, Gated Recurrent Unit (GRU) network, or any appropriate recurrent architecture. By analyzing the spatial feature vectors across multiple frames, the RNN processes temporal sequences to identify patterns in droplet movement or flow rate over time. The combination of the CNN and RNN improves free water detection accuracy and enhances robustness against noise or transient environmental variations.

The machine learning model generates output data identifying the free water droplets in the image, including spatial feature vectors representing characteristics of the detected droplets, such as shape and size. The machine learning model performs temporal analysis of the free water droplet movement, generating outputs such as motion trajectories, flow rate patterns, or other outputs representing patterns in free water droplet motion.

The data processing system applies post-processing techniques to the output of the trained machine learning model to enhance accuracy and refine detection results. The post-processing techniques used may include, but are not limited to, outlier removal to filter out the false positive detections, droplet aggregation analysis, volume estimation, time-series analysis, and confidence scoring.

For volume estimation of each free water droplet, the data processing system assumes that detected free water droplets are approximately spherical, allowing the volume of each free water droplet to be calculated based on their measured diameter. The data processing system performs droplet aggregation analysis to calculate a more stable and accurate water concentration. Droplet aggregation analysis is performed by grouping and aggregating the detected free water droplets over the time series of SWIR images or video frames to compute a total free water volume, which is then used to calculate the free water concentration of the analyzed volume of liquid. For example, the data processing system determines the concentration of free water by computing the ratio of the total free water volume to the total liquid volume analyzed over the time series. The water concentration is calculated in ppm.

The data processing system determines if the calculated free water concentration exceeds a threshold free water concentration. The free water threshold can be a user-defined value or dynamically determined based on operating conditions or historical sensor data. In response to determining that the free water concentration in the liquid exceeds the threshold free water concentration, the data processing system performs one or more mitigating actions. For example, the data processing system can generate and transmit a signal to an alarm system 310 that triggers audible alarms (e.g., sirens), flashing lights, or both.

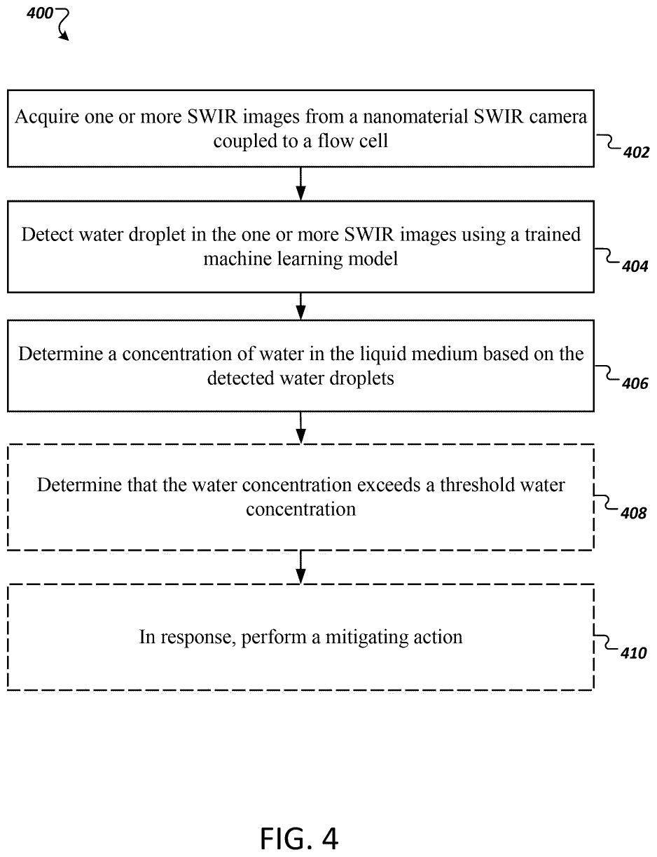

FIG. 4 is a flowchart for an example method 400 for measuring water concentration in a liquid medium using the systems of FIG. 1 or 2. The method 400 can be implemented on a data processing system such as a computer or control system (e.g., data processing system 120 or data processing system 218).

The data processing system acquires 402 one or more SWIR images from a nanomaterial-based SWIR camera coupled to a flow cell. The flow cell serves as a conjunction between two pipes which a liquid flows through. The liquid can include free water, hydrocarbon condensates, C3+ natural gas liquids, or all. With reference to FIG. 3, the one or more SWIR images can be a time series of images or frames extracted from a video which depict the liquid flowing through a pipe. The data processing system pre-processes the one or more SWIR images to remove unwanted artifacts from the SWIR images to prepare them for analysis by the trained machine learning model. The data processing system provides the pre-processed images to the trained machine learning model, which detects and measures the free water content in the liquid.

The data processing system detects 404 water droplets in the one or more SWIR images using a trained machine learning model. As described in FIG. 3, the trained machine learning model as implemented as a combination of a CNN and an RNN. The CNN processes the one or more SWIR images to generate a plurality of spatial feature vectors which encode characteristics of detected free water droplets, which are processed by the RNN to identify patterns in droplet movement or flow rate over time. The machine learning model generates output data identifying the free water droplets in the image, including spatial feature vectors representing characteristics of the detected droplets, such as shape and size. The machine learning model performs temporal analysis of the free water droplet movement, generating outputs such as motion trajectories, flow rate patterns, or other outputs representing patterns in free water droplet motion.

The data processing system applies post-processing techniques to the output of the trained machine learning model to refine the model's output to improve accuracy. The data processing system performs outlier removal to filter out the false positive detections. The data processing system performs droplet aggregation analysis, volume estimation, time-series analysis, and confidence scoring.

The data processing system determines 406 a concentration of water in the liquid medium based on the detected water droplets. The data processing system performs droplet aggregation analysis to calculate a more stable and accurate water concentration. Droplet aggregation analysis is performed by assuming that the detected free water droplets are approximately spherical, allowing the volume of each free water droplet to be calculated based on their measured diameters. The data processing system groups and aggregates these free water droplet volume estimations across the time series of SWIR images or video frames to compute a total free water volume, which is then used to determine the free water concentration of the analyzed volume of liquid.

In some embodiments, the data processing system determines 408 that the water concentration exceeds a threshold water concentration. The free water threshold can be a user defined value or dynamically determined based on operating conditions or historical sensor data.

In response to determining that the water concentration exceeds the threshold water concentration, the data processing system performs 410 a mitigating action. The data processing system can generate and transmit a signal to an alarm system 310 that triggers audible alarms (e.g., sirens), flashing lights, or both. The data processing system can also be configured to transmit a signal to other computers or processing devices to cause the other devices to generate the alert, such as a pop-up message on a display device. In some embodiments, the data processing system can generate commands to control fluid separation equipment to separate water from the liquid medium. For example, the data processing system can generate commands to control valves to direct the liquid medium to flow to equipment for further fluid separation. The measurement of free water concentration can serve as a quality control factor, and the data processing system can divert the liquid medium having excess water (e.g., the concentration of free water exceeds the threshold free water concentration) to processing equipment to process the liquid medium.

The data processing system trains the machine learning model with a training dataset including annotated SWIR images or videos of free water droplets under controlled conditions. The dataset may contain SWIR images or videos depicting various free water droplet sizes, shapes, and flow conditions to improve model generalization. Prior to training, the data processing system applies pre-processing techniques to the SWIR images or videos, such as noise reduction and augmentation to remove unwanted artifacts that can interfere with analysis, enhance image clarity, and improving model performance. The trained machine learning model is then validated using real-world data to ensure accurate detection of free water droplets in different operational environments.

The data processing system periodically retrains the trained machine learning model on real-world data obtained from the SWIR camera, visible light camera, or other sensor measurements. In some embodiments, retraining is performed based on a predefined schedule. In some embodiments, retraining is triggered automatically based on factors such as significant changes in environmental conditions, sensor degradation, or the availability of new data that highlights previously unseen patterns. To detect when retraining is needed, the system monitors several factors. The factors may include a drift in sensor data, such as a shift in baseline or increased noise levels which may indicate sensor degradation, a decreased detection accuracy which is evidenced by an increase in false positives or false negatives; and novel data patterns not previously encountered.

The data processing system retrains the machine learning model based on the extent of change in data distribution. In the case of severe sensor degradation or environmental changes that result in a significant data distribution shift, the data processing system retrains the machine learning model from scratch. If the data distribution shift is minor, the data processing system updates the machine learning model using incremental learning methods, such as fine-tuning.

In implementations including a SWIR camera and a visible light camera, the data processing system employs a multispectral fusion algorithm. The multispectral fusion algorithm combines the visible-light images from the CCD sensor and the SWIR images from the nanomaterial-based SWIR camera, captured simultaneously with the same or overlapping fields of view, to generate fused multispectral images. The fused multispectral images generated by this process retain spectral information from both the visible and SWIR ranges, enhancing the distinction between free water droplets and the liquid medium. These fused images are then provided to the trained machine learning model, which processes them using the same techniques applied to the SWIR images and frames as described above, extracting spatial and temporal features to improve free water droplet detection and concentration estimation. The machine learning model is trained on a dataset including fused multispectral images obtained from the SWIR camera and the visible light camera under controlled conditions.

In some implementations, the data processing system continuously acquires images from the SWIR camera and/or the visible light camera. For example, the data processing system acquires an image at a fixed rate (e.g., 1 or more image every 10 seconds, 1 or more image per second, 30 or more images per second).

In some implementations, the data processing system processes the SWIR images in real time. Real-time or near real-time processing and/or communication refers to a scenario in which received data (e.g., SWIR images) are processed as made available to systems and devices requesting those data immediately (e.g., within milliseconds, tens of milliseconds, or hundreds of milliseconds) after the processing of those data are completed, without introducing data persistence or store-then-forward actions. In this context, a real-time data processing system is configured to process a SWIR image or a visible light image as it arrives and determine the concentration of free water in the liquid as quickly as possible (though processing latency may occur). Though data can be buffered between module interfaces in a pipelined architecture, each individual module operates on the most recent data available to it. The overall result is a workflow that, in a real-time context, receives a data stream (e.g., SWIR images) and outputs processed data (e.g., free water concentration) based on that data stream in a first-in, first out manner. However, non-real-time contexts are also possible, in which data are stored (either in memory or persistently) for processing at a later time. In this context, modules of the data processing system do not necessarily operate on the most recent data available.

The system performs an automatic calibration process using real-time data from the SWIR camera, visible camera, and other sensors. The calibration process leverages the absorption properties of water in the SWIR range to account for variables such as flow rate, droplet size, and environmental conditions. The system may perform the calibration through a combination of hardware tuning (e.g., optics, sensors) and software adjustments (e.g., machine learning model refinements). This process ensures that the system remains accurate under different operating conditions and adapts dynamically over time.

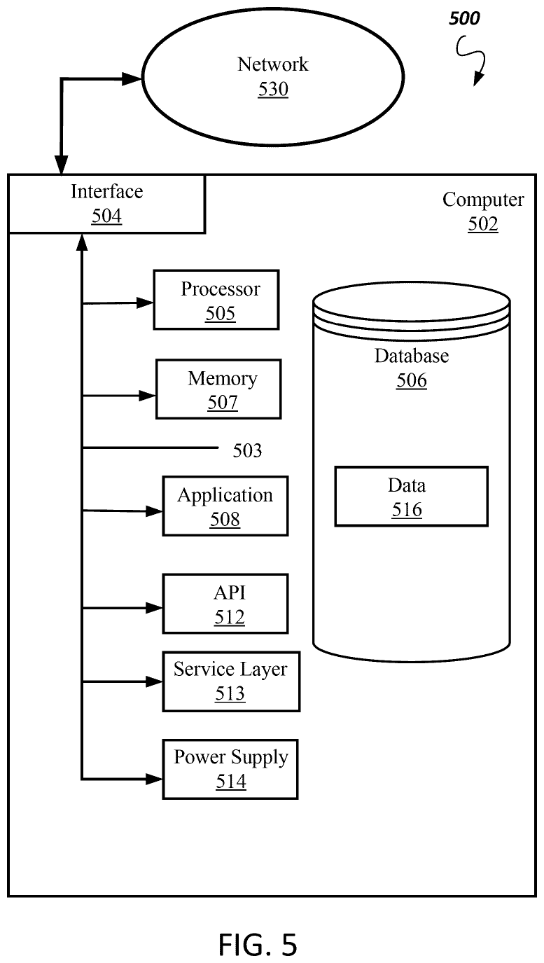

FIG. 5 is a block diagram of an example computer system 500 used to provide computational functionalities associated with described algorithms, methods, functions, processes, flows, and procedures described in the present disclosure, according to some implementations of the present disclosure. The illustrated computer 502 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 502 can include input devices such as keypads, keyboards, and touch screens that can accept user information. Also, the computer 502 can include output devices that can convey information associated with the operation of the computer 502. The information can include digital data, visual data, audio information, or a combination of information. The information can be presented in a graphical user interface (UI) (or GUI).

The computer 502 can serve in a role as a client, a network component, a server, a database, a persistency, or components of a computer system for performing the subject matter described in the present disclosure. The illustrated computer 502 is communicably coupled with a network 530. In some implementations, one or more components of the computer 502 can be configured to operate within different environments, including cloud-computing-based environments, local environments, global environments, and combinations of environments.

At a high level, the computer 502 is an electronic computing device operable to receive, transmit, process, store, and manage data and information associated with the described subject matter. According to some implementations, the computer 502 can also include, or be communicably coupled with, an application server, an email server, a web server, a caching server, a streaming data server, or a combination of servers.

The computer 502 can receive requests over network 530 from a client application (for example, executing on another computer 502). The computer 502 can respond to the received requests by processing the received requests using software applications. Requests can also be sent to the computer 502 from internal users (for example, from a command console), external (or third) parties, automated applications, entities, individuals, systems, and computers.

Each of the components of the computer 502 can communicate using a system bus 503. In some implementations, any, or all of the components of the computer 502, including hardware or software components, can interface with each other or the interface 504 (or a combination of both), over the system bus 503. Interfaces can use an application programming interface (API) 512, a service layer 513, or a combination of the API 512 and service layer 513. The API 512 can include specifications for routines, data structures, and object classes. The API 512 can be either computer-language independent or dependent. The API 512 can refer to a complete interface, a single function, or a set of APIs.

The service layer 513 can provide software services to the computer 502 and other components (whether illustrated or not) that are communicably coupled to the computer 502. The functionality of the computer 502 can be accessible for all service consumers using this service layer. Software services, such as those provided by the service layer 513, can provide reusable, defined functionalities through a defined interface. For example, the interface can be software written in JAVA, C++, or a language providing data in extensible markup language (XML) format. While illustrated as an integrated component of the computer 502, in alternative implementations, the API 512 or the service layer 513 can be stand-alone components in relation to other components of the computer 502 and other components communicably coupled to the computer 502. Moreover, any or all parts of the API 512 or the service layer 513 can be implemented as child or sub-modules of another software module, enterprise application, or hardware module without departing from the scope of the present disclosure.

The computer 502 includes an interface 504. Although illustrated as a single interface 504 in FIG. 5, two or more interfaces 504 can be used according to particular needs, desires, or particular implementations of the computer 502 and the described functionality. The interface 504 can be used by the computer 502 for communicating with other systems that are connected to the network 530 (whether illustrated or not) in a distributed environment. Generally, the interface 504 can include, or be implemented using, logic encoded in software or hardware (or a combination of software and hardware) operable to communicate with the network 530. More specifically, the interface 504 can include software supporting one or more communication protocols associated with communications. As such, the network 530 or the interface's hardware can be operable to communicate physical signals within and outside of the illustrated computer 502.

The computer 502 includes a processor 505. Although illustrated as a single processor 505 in FIG. 5, two or more processors 505 can be used according to particular needs, desires, or particular implementations of the computer 502 and the described functionality. Generally, the processor 505 can execute instructions and can manipulate data to perform the operations of the computer 502, including operations using algorithms, methods, functions, processes, flows, and procedures as described in the present disclosure.

The computer 502 also includes a database 506 that can hold data for the computer 502 and other components connected to the network 530 (whether illustrated or not). For example, database 506 can hold data 516. For example, database 506 can be an in-memory, conventional, or a database storing data consistent with the present disclosure. In some implementations, database 506 can be a combination of two or more different database types (for example, hybrid in-memory and conventional databases) according to particular needs, desires, or particular implementations of the computer 502 and the described functionality. Although illustrated as a single database 506 in FIG. 5, two or more databases (of the same, different, or combination of types) can be used according to particular needs, desires, or particular implementations of the computer 502 and the described functionality. While database 506 is illustrated as an internal component of the computer 502, in alternative implementations, database 506 can be external to the computer 502.

The computer 502 also includes a memory 507 that can hold data for the computer 502 or a combination of components connected to the network 530 (whether illustrated or not). Memory 507 can store any data consistent with the present disclosure. In some implementations, memory 507 can be a combination of two or more different types of memory (for example, a combination of semiconductor and magnetic storage) according to particular needs, desires, or particular implementations of the computer 502 and the described functionality. Although illustrated as a single memory 507 in FIG. 5, two or more memories 507 (of the same, different, or combination of types) can be used according to particular needs, desires, or particular implementations of the computer 502 and the described functionality. While memory 507 is illustrated as an internal component of the computer 502, in alternative implementations, memory 507 can be external to the computer 502.

The application 508 can be an algorithmic software engine providing functionality according to particular needs, desires, or particular implementations of the computer 502 and the described functionality. For example, application 508 can serve as one or more components, modules, or applications. Further, although illustrated as a single application 508, the application 508 can be implemented as multiple applications 508 on the computer 502. In addition, although illustrated as internal to the computer 502, in alternative implementations, the application 508 can be external to the computer 502.

The computer 502 can also include a power supply 514. The power supply 514 can include a rechargeable or non-rechargeable battery that can be configured to be either user-or non-user-replaceable. In some implementations, the power supply 514 can include power-conversion and management circuits, including recharging, standby, and power management functionalities. In some implementations, the power-supply 514 can include a power plug to allow the computer 502 to be plugged into a wall socket or a power source to, for example, power the computer 502 or recharge a rechargeable battery.

There can be any number of computers 502 associated with, or external to, a computer system containing computer 502, with each computer 502 communicating over network 530. Further, the terms “client,” “user,” and other appropriate terminology can be used interchangeably, as appropriate, without departing from the scope of the present disclosure. Moreover, the present disclosure contemplates that many users can use one computer 502 and one user can use multiple computers 502.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware-or software-based (or a combination of both hardware-and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example LINUX, UNIX, WINDOWS, MAC OS, ANDROID, or IOS.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of implementations of these systems and methods have been described. Nevertheless, it will be understood that various modifications may be made without departing from the spirit and scope of this disclosure. Accordingly, other implementations are within the scope of the following claims.

### Examples

In an example implementation, a system for measuring water concentration in a liquid medium includes a flow cell configured to fluidly couple to a pipe; a nanomaterial-based shortwave infrared (SWIR) camera coupled to a first side of the flow cell, the nanomaterial-based SWIR camera comprising a modular enclosure that houses a nanomaterial-based SWIR sensor, a passive cooling system, one or more interchangeable lenses, and a nanomaterial-based SWIR sensor. The system further comprising a light source coupled to a second side of the flow cell opposite the first side, one or more processors, and a memory unit storing instructions that when executed by the one or more processors cause the one or more processors to perform operations including acquiring one or more SWIR images captured by the nanomaterial-based SWIR camera, applying a preprocessing operation to the one or more SWIR images; detecting water droplets in the one or more SWIR images using a trained machine learning model; determining a concentration of water in the liquid medium based on the detected water droplets; and responsive to determining the concentration of water is above a threshold, performing a mitigating action.

In an aspect combinable with the example implementation, the nanomaterial-based SWIR sensor comprising quantum dots configured to detect SWIR light, wherein the quantum dots are selected from Indium Arsenide (InAs) quantum dots, Lead Sulfide (PbS) quantum dots, or a combination thereof.

In another aspect combinable with any of the previous aspects, the trained machine learning model comprises a Convolutional Neural Network (CNN) and a Recurrent Neural Network (RNN).

In another aspect combinable with any of the previous aspects, the operations include training the machine learning model using a training dataset comprising annotated SWIR images of water droplets in the liquid medium.

In another aspect combinable with any of the previous aspects, the preprocessing operation includes at least one of noise reduction and augmentation.

In another aspect combinable with any of the previous aspects, the trained machine learning model is retrained based on at least one retraining trigger, the retraining trigger including significant changes in environmental conditions, sensor degradation, or availability of new data indicating previously unobserved patterns.

In another aspect combinable with any of the previous aspects, performing the mitigating action comprises generating alerts, generating audible alarms, generating a pop-up message on a display, and transmitting signals to one or more other devices to automatically trigger one or more actions, the actions comprising controlling valves or directing a flow of the liquid medium, or any combinations thereof.

In another example implementation, a method for measuring water concentration in a liquid medium includes acquiring one or more SWIR images captured by the nanomaterial-based SWIR camera, applying a preprocessing operation to the one or more SWIR images; detecting water droplets in the one or more SWIR images using a trained machine learning model; determining a concentration of water in the liquid medium based on the detected water droplets; and responsive to determining the concentration of water is above a threshold, performing a mitigating action.

In an aspect combinable with the example implementation, the trained machine learning model comprises a Convolutional Neural Network (CNN) and a Recurrent Neural Network (RNN).

In another aspect combinable with any of the previous aspects, the operations include training the machine learning model using a training dataset comprising annotated SWIR images of water droplets in the liquid medium.

In another aspect combinable with any of the previous aspects, the preprocessing operation includes at least one of noise reduction and augmentation.

In another aspect combinable with any of the previous aspects, the trained machine learning model is retrained based on at least one retraining trigger, the retraining trigger including significant changes in environmental conditions, sensor degradation, or availability of new data indicating previously unobserved patterns.

In another aspect combinable with any of the previous aspects, performing the mitigating action comprises generating alerts, generating audible alarms, generating a pop-up message on a display, and transmitting signals to one or more other devices to automatically trigger one or more actions, the actions comprising controlling valves or directing a flow of the liquid medium, or any combinations thereof.

In another aspect combinable with any of the previous aspects, performing the mitigating action comprises generating alerts, generating audible alarms, generating a pop-up message on a display, and transmitting signals to one or more other devices to automatically trigger one or more actions, the actions comprising controlling valves or directing a flow of the liquid medium, or any combinations thereof.

In another aspect combinable with any of the previous aspects, applying a multi-spectral fusion algorithm to combine SWIR images obtained from the SWIR camera and images obtained from a CCD camera to generate a fused image.

In another example implementation, one or more non-transitory machine readable storage devices storing instructions for measuring water concentration in a liquid medium, the instructions being executable by one or more processors to cause performance of operations including includes acquiring one or more SWIR images captured by the nanomaterial-based SWIR camera, applying a preprocessing operation to the one or more SWIR images; detecting water droplets in the one or more SWIR images using a trained machine learning model; determining a concentration of water in the liquid medium based on the detected water droplets; and responsive to determining the concentration of water is above a threshold, performing a mitigating action.

In an aspect combinable with the example implementation, the trained machine learning model comprises a Convolutional Neural Network (CNN) and a Recurrent Neural Network (RNN).

In another aspect combinable with any of the previous aspects, the operations include training the machine learning model using a training dataset comprising annotated SWIR images of water droplets in the liquid medium.

In another aspect combinable with any of the previous aspects, the preprocessing operation includes at least one of noise reduction and augmentation.

In another aspect combinable with any of the previous aspects, the trained machine learning model is retrained based on at least one retraining trigger, the retraining trigger including significant changes in environmental conditions, sensor degradation, or availability of new data indicating previously unobserved patterns.

In another aspect combinable with any of the previous aspects, performing the mitigating action comprises generating alerts, generating audible alarms, generating a pop-up message on a display, and transmitting signals to one or more other devices to automatically trigger one or more actions, the actions comprising controlling valves or directing a flow of the liquid medium, or any combinations thereof.

What is claimed is:

1. A system for measuring water concentration in a liquid medium, the system comprising:
a flow cell configured to fluidly couple to a pipe;
a nanomaterial-based shortwave infrared (SWIR) camera coupled to a first side of the flow cell, wherein the nanomaterial-based SWIR camera comprises:
a nanomaterial-based SWIR sensor;
a passive cooling system;
one or more interchangeable lenses; and
a modular enclosure that houses the nanomaterial-based SWIR sensor, the passive cooling system, and the one or more interchangeable lenses;
a light source coupled to a second side of the flow cell opposite the first side;
one or more processors; and
a memory unit storing instructions that when executed by the one or more processors cause the one or more processors to perform operations comprising:
acquiring one or more SWIR images captured by the nanomaterial-based SWIR camera;
applying a preprocessing operation to the one or more SWIR images;
detecting water droplets in the one or more SWIR images using a trained machine learning model, wherein the trained machine learning model comprises a Convolutional Neural Network (CNN) and a Recurrent Neural Network (RNN);
determining a concentration of water in the liquid medium based on the detected water droplets; and
responsive to determining the concentration of water is above a threshold, performing a mitigating action.

2. The system of claim 1, the nanomaterial-based SWIR sensor comprising quantum dots configured to detect SWIR light, wherein the quantum dots are selected from Indium Arsenide (InAs) quantum dots, Lead Sulfide (PbS) quantum dots, or a combination thereof.

3. The system of claim 1, wherein the CNN extracts spatial feature vectors from the one or more SWIR images, the spatial feature vectors representing characteristics of the detected water droplets, and the RNN processes the spatial feature vectors to identify temporal patterns associated with the detected water droplets.

4. The system of claim 1, further comprising training the machine learning model using a training dataset comprising annotated SWIR images of water droplets in the liquid medium.

5. The system of claim 1, wherein the preprocessing operation includes at least one of noise reduction and augmentation.

6. The system of claim 1, wherein the trained machine learning model is retrained based on at least one retraining trigger, the retraining trigger including significant changes in environmental conditions, sensor degradation, or availability of new data indicating previously unobserved patterns.

7. The system of claim 1, wherein performing the mitigating action comprises generating alerts, generating audible alarms, generating a pop-up message on a display, and transmitting signals to one or more other devices to automatically trigger one or more actions, the actions comprising controlling valves or directing a flow of the liquid medium, or any combinations thereof.

8. A method for measuring water concentration in a liquid medium, the method comprising:
acquiring one or more nanomaterial-based shortwave infrared (SWIR) images captured by a nanomaterial-based SWIR camera;
applying a preprocessing operation to the one or more SWIR images;
detecting water droplets in the one or more SWIR images using a trained machine learning model, wherein the trained machine learning model comprises a Convolutional Neural Network (CNN) and a Recurrent Neural Network (RNN);
determining a concentration of water in the liquid medium based on the detected water droplets; and
responsive to determining the concentration of water is above a threshold, performing a mitigating action.

9. The method of claim 8, wherein the CNN extracts spatial feature vectors from the one or more SWIR images, the spatial feature vectors representing characteristics of the detected water droplets, and the RNN processes the spatial feature vectors to identify temporal patterns associated with the detected water droplets.

10. The method of claim 8, further comprising training the machine learning model using a training dataset comprising annotated SWIR images of water droplets in the liquid medium.

11. The method of claim 8, wherein the preprocessing operation includes at least one of noise reduction and augmentation.

12. The method of claim 8, wherein the trained machine learning model is retrained based on at least one retraining trigger, the retraining trigger including significant changes in environmental conditions, sensor degradation, or availability of new data indicating previously unobserved patterns.

13. The method of claim 8, wherein performing the mitigating action comprises generating alerts, generating audible alarms, generating a pop-up message on a display, and transmitting signals to one or more other devices to automatically trigger one or more actions, the actions comprising controlling valves or directing a flow of the liquid medium, or any combinations thereof.

14. The method of claim 8, further comprising applying a multi-spectral fusion algorithm to combine SWIR images obtained from the SWIR camera and images obtained from a Charge-Coupled Device (CCD) camera to generate a fused image.

15. One or more non-transitory machine-readable storage devices storing instructions for measuring water concentration in a liquid medium, the instructions being executable by one or more processors to cause performance of operations comprising:
acquiring one or more nanomaterial-based shortwave infrared (SWIR) images captured by a nanomaterial-based SWIR camera;
applying a preprocessing operation to the one or more SWIR images;
detecting water droplets in the one or more SWIR images using a trained machine learning model, wherein the trained machine learning model comprises a Convolutional Neural Network (CNN) and a Recurrent Neural Network (RNN);
determining a concentration of water in the liquid medium based on the detected water droplets; and
responsive to determining the concentration of water is above a threshold, performing a mitigating action.

16. The one or more non-transitory machine-readable storage devices of claim 15, wherein the CNN extracts spatial feature vectors from the one or more SWIR images, the spatial feature vectors representing characteristics of the detected water droplets, and the RNN processes the spatial feature vectors to identify temporal patterns associated with the detected water droplets.

17. The one or more non-transitory machine-readable storage devices of claim 15, further comprising training the machine learning model using a training dataset comprising annotated SWIR images of water droplets in the liquid medium.

18. The one or more non-transitory machine-readable storage devices of claim 15, wherein the preprocessing operation includes at least one of noise reduction and augmentation.

19. The one or more non-transitory machine-readable storage devices of claim 15, wherein the trained machine learning model is retrained based on at least one retraining trigger, the retraining trigger including significant changes in environmental conditions, sensor degradation, or availability of new data indicating previously unobserved patterns.

20. The one or more non-transitory machine-readable storage devices of claim 15, wherein performing the mitigating action comprises generating alerts, generating audible alarms, generating a pop-up message on a display, and transmitting signals to one or more other devices to automatically trigger one or more actions, the actions comprising controlling valves or directing a flow of the liquid medium, or any combinations thereof.
